(19) United States
(12) Patent Application Publication
(10) Pub. No.: US 2015/0093351 A1
(43) Pub. Date: Apr. 2, 2015

(54) MALODOR CONTROL COMPOSITIONS HAVING ACTIVATED ALKENES AND METHODS THEREOF

(71) Applicant: The Procter & Gamble Company, Cincinnati, OH (US)

(72) Inventors: Steven Anthony HORENZIAK, Cincinnati, OH (US); Cahit EYLEM, West Chester, OH (US); Michael-Vincent Nario MALANYAON, Indian Springs, OH (US)

(21) Appl. No.: 14/566,834

(22) Filed: Dec. 11, 2014

Related U.S. Application Data
(63) Continuation of application No. PCT/US2013/045874, filed on Jun. 14, 2013.
Application No. PCT/US2013/045874 is a continuation-in-part of application No. 13/524,042, filed on Jun. 15, 2012.

(51) Int. Cl. A61L 9/01

(52) U.S. Cl. CPC A61L 9/01
USPC 424/76.1

(57) ABSTRACT

A malodor control composition having activated alkenes and methods thereof are provided. In some embodiments, a catalyst is provided in the composition. The composition is suitable for a variety of applications, including use in fabric and air freshening products.

### FIELD OF THE INVENTION

The present invention relates to a malodor control composition having activated alkenes, and methods thereof. The malodor control composition is suitable for use in a variety of applications, including use in fabric and air freshening products.

### BACKGROUND OF THE INVENTION

Malodors can be associated with thiols, aldehydes, amines, sulfides, fatty acids, and alcohols. The difficulty in controlling malodors has spawned a diverse assortment of products to neutralize, block, mask, or contain malodors. There remains a need for a malodor control composition that neutralizes a broad range of malodors, without an overwhelming scent.

### SUMMARY OF THE INVENTION

In one embodiment, there is provided a malodor control composition comprising an activated alkene, and organic catalyst, wherein the composition is substantially free of mercaptans.

In another embodiment, there is provided a malodor control composition comprising an activated alkene; an organic catalyst; a surfactant; and an aqueous carrier, wherein the aqueous carrier is present in an amount greater than 50% to about 99.5%, by weight of the composition.

In yet another embodiment, there is provided a malodor control composition comprising from about 0.05% to about 25%, by weight of said composition, of an activated alkene; from 1% to about 10%, by weight of the malodor control composition, of an organic catalyst; and a polyamine polymer.

In yet another embodiment, there is provided a method of neutralizing a malodor in the air or on an inanimate surface comprising contacting said malodor with a composition comprising an activated alkene and an organic catalyst.

### DETAILED DESCRIPTION OF THE INVENTION

The present invention relates to a malodor control composition having activated alkenes for neutralizing malodors, and methods thereof. In some embodiments, the reaction between the activated alkene in the malodor control composition and the malodorous compound may be catalyzed by radical, cationic, or nucleophillic initiation.

### I. Malodor Control Composition

The malodor control composition includes an activated alkene and is designed to deliver genuine malodor neutralization and not function merely by covering up or masking odors. “Malodor” refers to compounds generally offensive or unpleasant to most people, such as the complex odors associated with bowel movements. “Neutralize” or “neutralization” refers to the ability of a compound or product to reduce or eliminate malodorous compounds. Odor neutralization may be partial, affecting only some of the malodorous compounds in a given context, or affecting only part of a malodorous compound. A malodorous compound may be neutralized by chemical reaction resulting in a new chemical entity, by sequestration, by chelation, by association, or by any other interaction rendering the malodorous compound less malodorous or non-malodorous. Odor neutralization may be distinguished from odor masking or odor blocking by a change in the malodorous compound, as opposed to a change in the ability to perceive the malodor without any corresponding change in the condition of the malodorous compound.

Genuine malodor neutralization provides a sensory and analytically measurable (e.g. gas chromatograph) malodor reduction. Thus, if the malodor control composition delivers a genuine malodor neutralization, the composition will reduce malodors in the vapor and/or liquid phase.

While the malodor control composition of the present invention may react with and neutralize odors, including thiol based odors, in the air and/or on inanimate surfaces once released into the air or onto the surface, in some embodiments, the composition is substantially free of mercaptans (e.g. compounds having a thiol functionality). A composition that is substantially free of mercaptans is one that has a mercaptan and activated alkene molar ratio of less than 1:2, alternatively the composition comprises less than 10% mercaptans, by weight of the composition, alternatively less than 5%, alternatively less than 3%. In some embodiments, the malodor composition is free of mercaptans.

A. Activated Alkenes

An activated alkene is a molecule that has at least one unsaturation with an electron withdrawing functionality adjacent to said unsaturation. In such a molecule, the unsaturation may be electron deficient, and therefore may have increased susceptibility to reaction with malodorous compounds, especially those containing a nucleophilic or anionic functionality.

An activated alkene has the general structure (I) shown below:

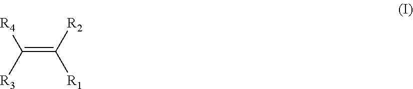

where at least one of R1, R2, R3 and R4 represents an electron withdrawing group. Up to four of R1, R2, R3 and R4 may be interconnected to form cyclic structures.

The electron withdrawing group may be, for example, a carbonyl or thiocarbonyl group, a carboxyl or thiocarboxyl group, an ester or thioester group, an amide, a nitro group, a nitrile group, a trihalide, a halide, a cyano group, a sulfonate group, or a phosphate group.

The unsaturation may comprise a double or triple bond.

Suitable activated alkenes include, but are not limited to, maleimides, acrylic acid, acrylic acid esters, methacrylic acid, methacylic acid esters, fumaric acid, fumaric acid esters, maleic acid, maleic acid esters, acrylonitriles and α, β unsaturated ketones and aldehydes.

In some embodiments, the molecule containing the electron deficient alkene may be water soluble for use in aqueous compositions like fabric freshening formulations. Suitable water soluble electron deficient alkenes are, for example, those containing ethylene glycol or polyethylene glycol (“PEG”) functionality, or esters of glycerol and acrylic acid derivatives, such as glycerol dimethacrylate.

In other embodiments, such as a vapor phase application (e.g. pluggable air freshener, Febreze® Set & Refresh air freshener, and other passive air freshening diffusers), the molecule containing the electron deficient alkene may have a vapor pressure (“VP”) range from about 0.001 to about 0.5, alternatively from about 0.01 to about 0.1 torr at 25° C. Suitable vapor phase electron deficient alkenes are, for example, diethyl maleate, diethyl fumarate, dibutyl maleate, dibutyl fumarate, acrylic acid esters containing less than about 18 carbon atoms, and enones with molecular weights less than about 300 Daltons including, for example, damascones, ionones, citral, maltols, and damascenones.

One suitable activated alkene is an α, β unsaturated carbonyl compound. In this case, at least one of R1, R2, R3 and R4 comprises a carbonyl group. The carbonyl group or groups may be present in the form of, for example, ketone, ester, aldehyde, amide or imide functionalities.

In one embodiment, the activated alkene is an α, β unsaturated carbonyl compound containing at least one unsaturation with at least 2 electron withdrawing carbonyl groups adjacent to the unsaturation. For example, this may include esters of fumaric or maleic acid, or derivatives of cis- or trans-2-butene 1,4 dione as shown in the structures below:

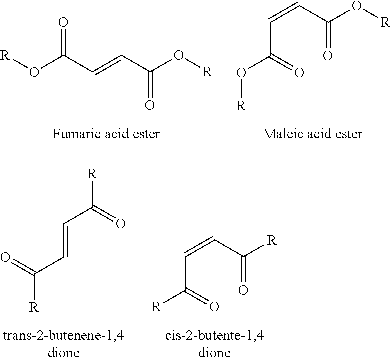

where R represents, for example, a hydrogen atom, an halide, a linear, branched or cyclic alkyl, alkenyl, alkynl or an aryl group which may be further substituted with other functionalities such as hydroxide, glycol, or carboxylic acid.

Another suitable example of an α, β unsaturated carbonyl compound containing at least one unsaturation with at least 2 electron withdrawing carbonyl groups adjacent to the unsaturation is a maleimide compound such as N-alkyl malemide as shown below:

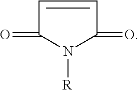

The N-alkyl group of the maleimide compound, represented by R, may be further substituted with other functionalities, for example, with PEG to impart a degree of water solubility to the maleimide compound. One example of a suitable PEG modified maleimide is methoxyl PEG maleimide:

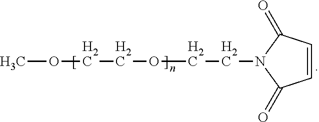

There is no limitation implied as to the value of n, or the molecular weight of such a suitable PEG modified maleimide.

Suitable maleimides also include bis-maleimides. One example of suitable bismaleimide is shown below:

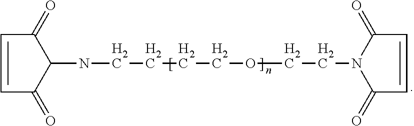

There is no limitation implied as to the value of n or the molecular weight of such a suitable bismaleimide.

Another suitable activated alkene is an α, β unsaturated carbonyl compound where the electron deficient unsaturation is in a chain terminal or “1” position), for example acrylic acid and methacrylic acid esters and diesters including, but not limited to hexyl acrylate and other alkyl acrylates, 1-6 hexanediol dimethacrylate, 1-3 glycerol dimethacrylate, and 2-hydroxyethyl methacrylate.

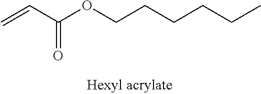

Another suitable activated alkene molecule contains both α, β unsaturated carbonyl and alcohol, glycol or polyglycol groups. Suitable activated alkenes of this type include, for example, PEG methacrylates, PEG dimethacrylates, glycerol dimethacrylate, 2-hydroxyethyl methacrylate, and triethylene glycol dimethacrylate.

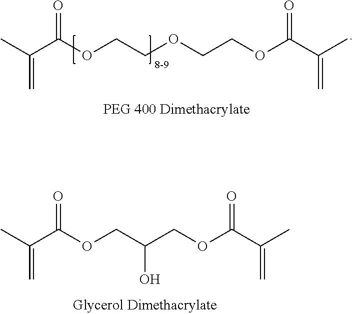

The electron deficient alkene may optionally be attached to a polymer, oligimer or other substrate such as silica, for example, 3-(maleimido)propyl functional silica gel:

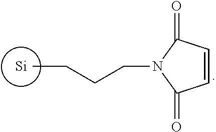

The activated alkene may be present in a malodor control composition in an effective amount to show analytically measurable malodor removal. The effective amount of activated alkene in the malodor control composition may be from about 0.0005% to about 100%, alternatively from about 0.005 to about 50%, alternatively from about 0.05% to about 25%, alternatively from about 0.05% to about 10%, alternatively from about 0.25% to about 1%, alternatively from about 0.25% to about 0.5%, by weight of the malodor control composition. The activated alkene present in the malodor control composition may comprise one suitable activated alkene or a mixture thereof.

B. Catalyst

The malodor control composition of the present invention may include a catalyst. The catalyst may comprise a nucleophile including, but not limited to, a primary amine, a secondary amine, a phosphine, or an imidazole functional compound. Non-limiting examples of suitable nucleophillic catalysts include primary n-alkyl amines up to C24 including n-hexyl amine and n-octylamine, and secondary alkyl amines including di-n-propyl amine. Other examples of suitable nucleophillic catalysts include trialkyl phosphines such as tri-n-propylphosphine.

In one embodiment, the organic catalyst comprises a primary amine, such as n-octyl amine, n-hexdyl amine, or n-decyl amine.

The catalyst may also be an organic or inorganic base, including bases with poor nucleophilicity, for example, amidines including a diazabicylo[5.4.0]undec-7-ene (“DBU”) or a diazabicyclo[4.3.0]non-5-ene (“DBN”) as shown below.

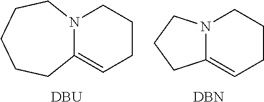

Other suitable bases include lithium diisopropylamide, N—N-diisopropylethylamine, tertiary amines including triethyl amine, and 1,4-diazabicyclo[2.2.2]octane.

The catalyst may be present in any amount. In one embodiment, the catalyst is present in a molar ratio of catalyst to activated alkene from about 1:1 to about 1:1,000,00, alternatively from about 1:5 to about 1:100,000, alternatively from about 1:10 to 1:250. The amount of catalyst in the composition may be from about 0.1% to about 50%, alternatively from about 0.5% to about 20%, alternatively, from about 1% to about 10%, by weight of the malodor control composition.

C. Polyamine Polymer

In aqueous compositions, the malodor control composition of the present invention may include a polyamine polymer having a general structure (II):

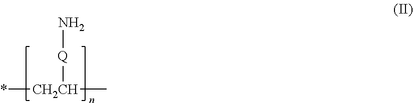

where Q is an integer having values between 0-3.

In one embodiment, the polymer includes a polyvinylamine (“PVam”) backbone. A PVam is a linear polymer with primary amine groups directly linked to the main chain of alternating carbons. Since the vinylamine monomer is not available (due to tautomerization equilibrium with acetaldehyde imine), PVam is usually manufactured from hydrolysis of poly(N-vinylformamide) (“PVNF”). In this process, formamide groups are readily hydrolyzed to primary amine groups as described by the following formula (IIa):

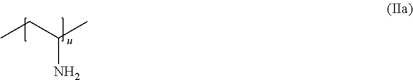

where n is the number of monomers present in the polymer and can range from 100 to 5000 depending on the molecular weight of PVNF used for hydrolysis. The degree of hydrolysis of PVNF can be used to produce adaptable copolymers of PVFA-co-PVAm with different formamide/amine ratios. For example, PVNF having a molecular weight of 10,000 with 50% hydrolysis will have n=141 consisting of 50% formamide and 50% primary amine groups.

PVams may be partially hydrolyzed meaning that 1% to 99%, alternatively 30% to 99%, alternatively 50% to 99%, alternatively 70% to 99%, alternatively 80% to 99%, alternatively 85% to 99%, alternatively 90% to 99%, alternatively 95% to 99%, alternatively 97% to 99%, alternatively 99% of the PVam is hydrolyzed. It has been found that high degree of hydrolysis of PVam increases the resulting polymer's ability to mitigate malodors.

PVams that can be hydrolyzed may have an average molecular weight (“MW”) of 5,000 to 350,000. Suitable hydrolyzed PVams are commercially available from BASF. Some examples include Lupamin™ 9095, 9030, 5095, and 1595. Such hydrolyzed PVams may then be hydrophobically modified. Hydrophobic modification, as described below may further improve malodor removal efficacy.

In another embodiment, the polymer includes a polyalkylenimine backbone. Polyalkylenimines include polyethyleneimines (“PEIs”) and polypropylenimines as well as the C4-C12 alkylenimines.

PEIs are a suitable polyalkylenimine. The chemical structure of a PEI follows a simple principle: one amine function and two carbons. PEIs have the following general formula (IIb):

—(CH₂—CH₂—NH)n (IIb)

where n=10-105.

PEIs constitute a large family of water-soluble polyamines of varying molecular weight, structure, and degree of modification. They may act as weak bases and may exhibit a cationic character depending on the extent of protonation driven by pH.

PEIs are produced by the ring-opening cationic polymerization of ethyleneimine as shown below.

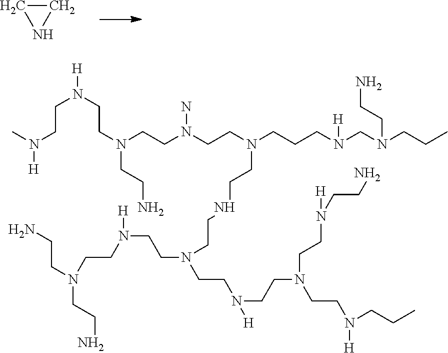

PEIs are believed to be highly branched containing primary, secondary, and tertiary amine groups in the ratio of about 1:2:1. PEIs may comprise a primary amine range from about 30% to about 40%, alternatively from about 32% to about 38%, alternatively from about 34% to about 36%. PEIs may comprise a secondary amine range from about 30% to about 40%, alternatively from about 32% to about 38%, alternatively from about 34% to about 36%. PEIs may comprise a tertiary amine range from about 25% to about 35%, alternatively from about 27% to about 33%, alternatively from about 29% to about 31%.

Other routes of synthesis may lead to products with a modified branched chain structure or even to linear chain PEIs. Linear PEIs contain amine sites in the main chain while the branched PEIs contain amines on the main and side chains. Below is an example of a linear PEI.

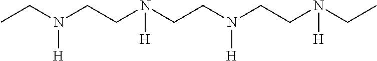

The composition of the present invention may comprise PEIs having a MW of about 800 to about 2,000,000, alternatively about 1,000 to about 2,000,000, alternatively about 1,200 to about 25,000, alternatively about 1,300 to about 25,000, alternatively about 2,000 to about 25,000, alternatively about 10,000 to about 2,000,000, alternatively about 25,000 to about 2,000,000, alternatively about 25,000.

In one embodiment, the PEI may have a specific gravity of 1.05 and/or an amine value of 18 (mmol/g, solid). For clarity, such specific gravity and/or amine value of the PEI describes the PEI before it is modified or added as part of an aqueous composition. One skilled in the art will appreciate, for example, the primary and secondary amino groups may react with other components of the composition.

Exemplary PEIs include those that are commercially available under the tradename Lupasol® from BASF or the tradename Epomine™ from Nippon Shokubia.

In some embodiments, less than 100% of the active amine sites on a polyamine polymer are substituted with hydrophobic functional groups, alternatively about 0.5% to about 90%, alternatively about 0.5% to about 80%, alternatively about 0.5% to about 70%, alternatively about 0.5% to about 60%, alternatively about 0.5% to about 50%, alternatively about 0.5% to about 40%, alternatively about 0.5% to about 35%, alternatively about 0.5% to about 30%, alternatively about 1% to about 30%, alternatively about 1% to about 25%, alternatively about 1% to about 20%, alternatively about 5% to about 20%, alternatively about 10% to about 30%, alternatively about 20% to about 30%, alternatively about 20% of the active amine sites are substituted with hydrophobic functional groups. When a polyamine polymer has active amine sites that are fully substituted with hydrophobic functional groups, such hydrophobically modified polyamine polymers (“HMPs”) may have no activity for malodor control.

Other non-limiting examples of polyamine polymers suitable in the present composition include polyamidoamines (“PAMams”), polyallyamines (“PAams”), polyetheramines (“PEams”), and mixtures thereof, or other nitrogen containing polymers, such as lysine, or mixtures of these nitrogen containing polymers.

Suitable levels of polyamine polymer or HMPs in the present composition are from about 0.01% to about 10%, alternatively from about 0.01% to about 2%, alternatively from about 0.01% to about 1%, alternatively from about 0.01% to about 0.8%, alternatively from about 0.01% to about 0.6%, alternatively from about 0.01% to about 0.1%, alternatively from about 0.01% to about 0.07%, alternatively about 0.07%, alternatively about 0.5%, by weight of the composition. Compositions with higher amounts of polymers may cause soiling or discoloration, and/or leave visible residue or stains on fabrics.

In certain embodiments, the polyamine polymer may be in the form of a metal coordinated complex with a transition metal ion such as zinc, silver, copper, or mixtures thereof. The metal coordinated complex comprises a metal and any modified or unmodified polymer disclosed herein, or mixtures thereof. Metal coordination may improve the odor neutralization of a malodor control polymer. Metal coordination might also provide reduction of malodor from microbial sources. Suitable metals that coordinate with such polymers include zinc, copper, silver, and mixtures thereof. Suitable metals also include Na, K, Ca, Mg, and non-transition metals, including Sn, Bi, and Al.

In some embodiments, the metal coordinated complex is a HMP having at least 5% of its primary, secondary, and/or tertiary amine sites left unmodified for not only malodor efficacy but also for metal binding capacity.

Metal coordinated complexes may have a metal and polymer weight ratio from 0.001 and 50, alternatively from 0.001 to 20, alternatively from 0.001 to 15, alternatively from 0.001 to 10, alternatively from 0.005 to 5.0, alternatively from 0.1 to 1.0, alternatively from 0.1 to 0.5, alternatively from 0.001 to 0.01.

In one embodiment, the composition includes a zinc polymer complex having a pH of 7. It is believed that at such pH the competition between protonation and metal coordination of amine sites provides a unique coordination environment for zinc. This unique bonding makes the zinc ions readily available for additional interactions with malodor molecules, while preventing the release of zinc ions from the metal coordinated complex.

### Water Solubility of Polyamine Polymer

This test illustrates the benchmarking ambient temperature water solubility of polymers against beta-cyclodextrin (1.8 g/100 ml) and hydroxypropyl modified beta cyclodextrin (60+g/100 ml). 1% water solubility is used as a screening criteria for polymers.

Room temperature equilibrium water solubility of polymers may be determined by adding weighed quantities of polymers into 100 ml of deionized water and allowing the added polymers to completely dissolve. This process is repeated until the added polymers are no longer soluble. Equilibrium water solubility is then calculated based on how much polymer is dissolved in 100 ml water.

TABLE 1

| | Equilibrium Water |
|---|---|
| | Solubility (g/100 ml |
| Polymer | water at 25° C.) |
| Lupasol G100 (PEI 5,000) | miscible at all levels (70+) |
| C6 modified PEI 1800 | 30+ |
| (0.25 C6/NH) | |
| Dodecene oxide modified PEI5,000 | ~24 |
| (0.1 dodecene oxide/NH) | |
| Dodecene oxide modified PEI5,000 | ~4 |
| (0.2 dodecene oxide/NH) | |
| Dodecene oxide modified PEI5,000 | <0.1 |
| (0.5 dodecene oxide/NH) | |
| Dodecene oxide modified PEI25,000 | ~21 |
| (0.1 dodecene oxide/NH) | |
| Dodecene oxide modified PEI25,000 | <0.1 |
| (0.2 dodecene oxide/NH) | |
| Dodecene oxide and ethylene oxide (EO) | ~6 |
| modified PEI25,000 | |
| (0.8 EO and 0.2 dodecene oxide/NH) | |

When the polymer is not water soluble (e.g. less than 0.05%), capping with a hydrophilic molecule may be desired to assist with water solubility. Suitable hydrophilic molecules include EO or other suitable hydrophilic functional groups.

D. Perfume Mixture

The malodor control composition may include a mixture of perfume raw materials such as volatile aldehydes, esters and/or alcohols. One or more of the perfume materials may comprise an activated alkene.

The malodor control composition may include perfume raw materials that provide a functional (e.g. malodor removal, assisting with volatilization of compounds) and/or a hedonic benefit (i.e. primarily present to provide a pleasant fragrance). Suitable perfumes are disclosed in U.S. Pat. No. 6,248,135, which is incorporated in its entirety by reference.

One embodiment of a perfume mixture is a low scent formula shown in Table 2.

TABLE 2
Low Scent Perfume Mixture

| Perfume Material | CAS Number | Wt. % |
|---|---|---|
| Floral Super | 71077-31-1 | 1 |
| Undecylenic Aldehyde | 112-45-8 | 0.5 |
| Pino Acetaldehyde | 33885-51-7 | 1 |
| Cedryl Methyl Ether | 19870-74-7 | 1 |
| Florhydral | 125109-85-5 | 10 |
| Cymal | 103-95-7 | 3 |
| Adoxal | 141-13-9 | 1 |
| Floralozone | 67634-14-4 | 12 |
| Bourgeonal | 18127-01-0 | 1.5 |
| Benzophenone | 119-61-9 | 20 |
| Vertofix Coeur | 32388-55-9 | 10 |
| Helional | 1205-17-0 | 15 |
| Methyl Palmitate | 112-39-0 | 4.8 |
| Vetivert Acetate | 68917-34-0 | 0.2 |
| Farnesol | 4602-84-0 | 15 |
| Flor Acetate | 2500-83-6 | 4 |

In other embodiments, the malodor control composition contains no perfume raw materials. While there may be some scent from certain constituents of the malodor control composition, in such embodiments, the composition is fragrance free.

In a water containing formulation, the perfume mixture can be formulated into the malodor control composition in any desired amount, for example, at about 1%, alternatively from about 0.01% to about 10%, alternatively from about 0.05% to about 5%, alternatively from about 0.5% to about 2%, by weight of the malodor control composition. For water-free malodor control compositions (i.e. vapor phase malodor control compositions), the perfume mixture may comprise any desired amount of the malodor control composition, for example, 20%, alternatively from about 5% to about 99%, alternatively from about 10% to about 50%, alternatively from about 15% to about 25%, by weight of the malodor control composition.

In some embodiments where volatility is not important for neutralizing a malodor, the present invention may include poly-aldehydes, for example, di-, tri-, tetra-aldehydes. Such embodiments may include laundry detergents, additive, and the like for leave-on, through the wash, and rinse-off type of applications.

E. Surfactants

The malodor control composition may comprise a surfactant. The surfactant may be selected from the group consisting of cationic, anionic, non-ionic, and mixtures thereof. In one embodiment, the surfactant is non-ionic. Non-limiting examples of suitable surfactants include ethoxylated hydrogenated castor oils, ethoxylated alcohols, and polyalkylene oxide polysiloxanes, and combinations thereof.

The surfactant is present in an effective amount to achieve dispersion or emulsification of the activated alkenes, perfume raw materials, or other materials in the malodor control composition. These effective amounts of surfactant may be, for example, less than about 3%, alternatively from about 0.01% to about 1%, alternatively from about 0.05% to about 0.5%, by weight of the composition.

F. Aqueous Carrier

The malodor control composition of the present invention may include an aqueous carrier. The aqueous carrier may be distilled, deionized, or tap water. Water may be present in an amount of greater than 50% to about 99.5%, alternatively from about 80% to about 99.5%, alternatively from about 92% to about 99.5%, alternatively about 95%, by weight of the composition. Water containing a small amount of low molecular weight monohydric alcohols, e.g., ethanol, methanol, and isopropanol, or polyols, such as ethylene glycol and propylene glycol, can also be useful. However, the volatile low molecular weight monohydric alcohols such as ethanol and/or isopropanol should be limited since these volatile organic compounds will contribute both to flammability problems and environmental pollution problems. If small amounts of low molecular weight monohydric alcohols are present in the composition of the present invention due to the addition of alcohols to such ingredients as perfumes and as stabilizers for some preservatives, the level of monohydric alcohol may be less than about 6%, alternatively less than about 3%, alternatively less than about 1%, by weight of the composition.

G. Solvents

The malodor control composition may contain one or more commercially available solvents. In one example, the solvent comprises ethyl alcohol or ethanol.

H. Other Optional Ingredients

The malodor control composition may, optionally, include one or more radical scavengers or antioxidants, such as butylhydroxytoluene (“BHT”), ascorbic acid, α-tocopherol, hydroquinone (“HQ”), or hydroquinone monomethyl ether (“MeHQ”). Further, the malodor control composition may optionally contain odor masking agents, odor blocking agents, and/or diluents. “Odor blocking” refers to the ability of a compound to dull the human sense of smell. “Odor-masking” refers to the ability of a compound to mask or hide a malodorous compound. Odor-masking may include a compound with a non-offensive or pleasant smell that is dosed such it limits the ability to sense a malodorous compound. Odor-masking may involve the selection of compounds which coordinate with an anticipated malodor to change the perception of the overall scent provided by the combination of odorous compounds.

For example, the malodor control composition may include perfume ionones and/or a diluents in any amount. For example, a diluent may be present in an amount from about 1% to about 99.5%, alternatively from about 50% to about 99.5%, alternatively greater than 50% to about 99.5%, alternatively from about 5% to about 50%, alternatively from about 10% to about 30%, by weight of the composition. Diluents with low scent intensity may be preferred, but are not required. Non-limiting exemplary diluents include DBE-LVP (mixed aliphatic ester fluid (CAS #1119-40-0 and CAS #627-93-0 from INVISTA)), glycol ethers such as dipropylene glycol monomethyl ether, tripropylene glycol methyl ether, dipropylene glycol n-propyl ether, or dipropylene glycol methyl ether acetate; 3-methoxy-3-methyl-1-butanol; esters such as isononyl acetate, diethyl adipate and dioctyl adipate; benzyl alcohol; florol; Xiameter® PMX-200 Silicone Fluid 1.5CS® (from Dow Corning Co.); cellulose; ethyl ether; ethylene glycol; triethylene glycol; and mixtures thereof.

### II. Methods of Use

The malodor control composition of the present invention may be used in a wide variety of applications to neutralize malodors in the air or on inanimate surface by contacting a malodor with effective amounts of said composition. In some embodiments, the malodor control composition may be formulated for use in energized vapor phase systems. “Energized” as used herein refers to a system that operates by using an electrical energy source, such as a battery or electrical wall outlet, to emit a targeted active. For such systems, the VP of activated alkenes and catalyst, when present, may be about 0.001 torr to about 20 torr, alternatively about 0.01 torr to about 10 torr, measured at 25° C. Example of energized vapor phase system include the liquid electric pluggable air freshening devices sold under the Febreze® Noticeables and AmbiPur® brands.

In some embodiments, the malodor control composition may be formulated for use in non-energized vapor phase systems. “Non-energized” as used herein refers to a system that emits a targeted active passively or without the need for an electrical energy source. Aerosol sprayers and traditional trigger/pump sprayers are considered non-energized systems. For such non-energized systems, the VP of the activated alkenes and catalyst, when present may be about 0.01 torr to about 20 torr, alternatively about 0.05 torr to about 10 torr, measured at 25° C. Non-limiting examples of a non-energized vapor phase system are passive air freshening diffusers such as those known by the trade name Renuzit® Crystal Elements; and aerosol sprays such as fabric and air freshening sprays and body deodorants.

In other embodiments, the malodor control composition may be formulated for use in a liquid phase system. For such systems, the VP of the activated alkenes and catalyst, when present, may be about 0 torr to about 20 torr, alternatively about 0.0001 torr to about 10 torr, measured at 25° C. Non-limiting examples of a liquid phase system are liquid laundry products such as laundry detergents and additives; dish detergents; personal hygiene products such as body washes, shampoos, conditioners. The malodor control composition may also be formulated for use in substrates such as plastics, wovens, or non-wovens (e.g cellulose fibers for paper products). Such substrates may be used as pet food packaging; paper towels; tissues; trash bags; diapers; baby wipes; adult incontinence products; feminine hygiene products such as sanitary napkins and tampons. The malodor control composition may also be formulated for use in commercial or industrial systems such as in septic tanks or sewage treatment equipment.

### EXAMPLES

### Example 1

### Butanethiol and Butylamine Removal by Activated Alkene

This example illustrates the malodor removal efficacy of an exemplary aqueous fabric refresher composition as shown in Table 3 (“Composition 3”). All activated alkenes are present in Composition 3 in an amount of 0.5 wt. %, except PEG 400 Dimethylacrylate which is present in Composition 3 in amount of 1.0 wt. %.

TABLE 3

| Ingredients | Wt. % |
|---|---|
| Ethanol | 3.0 |
| Surfactant (Silwet 7600) | 0.1 |
| Activated Alkene | 0.5 |
| Maleic acid | as needed for |
| | composition to |
| | reach pH 7 |
| Water | balance |

Composition 3 is prepared with each activated alkene shown in Table 4. In addition, a Control composition is prepared according to the composition in Table 3, except the activated alkene is omitted. Composition 3 with each activated alkene and the Control composition are then tested for malodor removal performance as described below.

n-Butanethiol and di-n-propoyl sulfide were chosen as chemical surrogates for sulfur-containing odors such as onion, garlic, sewage, etc. n-Butylamine was used as a representative for amine-containing odors such as fish, pet urine, etc.

5 ml of Composition 3 and the Control composition are placed into a separate GC-MS vial and each spiked with 5 microliters of butanthiol or butylamine. The compositions are first equilibrated at room temperature for 2 hours, then incubated at 35° C. for 30 minutes. The headspace of each vial is finally sampled using a polydimethyl siloxane (“PDMS”)/SPME fiber and analyzed by GC/MS. The head space concentrations of odor molecules are measured and the data are normalized to the Control composition.

Results of the GC/MS analysis are shown in Table 4. Numbers less than 1 denote reduced levels of malodor molecules present in the headspace of Composition 3 relative to the Control composition. The reduced malodor level is attributed to high malodor control efficacy of Composition 3.

TABLE 4

| | Butylamine | | Butanethiol | |
|---|---|---|---|---|
| | Repli- | Repli- | Repli- | Repli- |
| | cate 1 | cate 2 | cate 1 | cate 2 |
| Control composition (no | 1.0 | 1.0 | 1.0 | 1.0 |
| activated alkene or catalyst) | | | | |
| Composition 3 with Activated | | | | |
| Alkene: | | | | |
| N,N Dimethyl Acrylamide | 1.0 | 1.0 | 0.8 | 0.8 |
| Ethyl Maltol | 0.0 | 0.0 | 0.8 | 0.9 |
| Diethyl Maleate | 0.5 | 0.0 | 0.7 | 0.6 |
| Damascenone | 1.0 | 1.1 | 0.6 | 0.7 |
| Diethyl Fumarate | 0.1 | 0.1 | 0.4 | 0.3 |
| Citral | 0.0 | 0.0 | 0.1 | 0.1 |
| Hexyl Acrylate | 0.6 | 0.5 | 0.1 | 0.1 |
| N-Cyclohexylmaleimide | 0.0 | 0.0 | 0.0 | 0.0 |
| N-propyl maleimido | 0.2 | 0.2 | 0.0 | 0.0 |
| grafted silica gel | | | | |
| PEG 400 Dimethylacrylate | — | — | 0.5 | 0.4 |
| (@1.0 wt %) | | | | |

### Example 2

### Butanethiol Removal by Activated Alkene and Catalyst

This example illustrates the malodor removal efficacy of an exemplary aqueous fabric refresher composition as shown in Table 5 (“Composition 5”).

TABLE 5

| Ingredients | Wt. % |
|---|---|
| Ethanol | 3.0 |
| Surfactant (Silwet 7600) | 0.1 |
| Activated Alkene + catalyst | 0.5 |
| Maleic acid | as needed to reach Ph 7 |
| Water | balance |

Final Ph = 7

Example 1 is repeated using Composition 5 which is prepared with each alkene-catalyst system shown in Table 6. The results of the SPME GCMS analysis are shown in Table 6. This demonstrates the effectiveness of the catalyst in improving the performance of the malodor control composition of the present invention.

TABLE 6

| | | Catalyst | | |
|---|---|---|---|---|
| | | n-octylamine | n-hexyl amine | DBU |
| | No | 1:15 molar ratio of | 1:15 molar ratio of | 1:150 molar ratio of |
| | catalyst | catalyst:alkene | catalyst:alkene | catalyst:alkene |
| Control (no activated | 1.0 | NA | NA | NA |
| alkene or catalyst) | | | | |
| Composition 5 with | | | | |
| Activated Alkene: | | | | |
| N,N Dimethyl | 0.8 | 0.7 | 0.6 | 0.3 |
| Acrylamide | | | | |
| Diethyl Maleate | 0.7 | 0.04 | 0.1 | 0.5 |
| Damascenone | 0.6 | 0.6 | 0.4 | 0.8 |
| Diethyl Fumarate | 0.3 | 0 | 0 | 0.2 |
| Hexyl Acrylate | 0.1 | 0 | 0.02 | 0.2 |

### Example 3

### Vapor-Phase Butanethiol Removal

This example illustrates the malodor removal efficacy of exemplary activated alkenes-catalyst systems in the vapor phase.

Malodor standards are prepared by pipeting 1.0 mL butanethiol (sulfur-based malodor) into a 1.2 liter gas sampling bag. The bag is then filled with 500 ml of nitrogen and then placed in an oven at 50° C. for 20 minutes and subsequently allowed to cool back to room temperature to ensure saturation of the butanethiol in the nitrogen headspace.

A 1 μL sample of each activated alkene-catalyst combination listed in Table 7 is pipeted into an individual 10 mL silanized headspace vial. The vials are sealed and allowed to equilibrate for at least 12 hours. This procedure is repeated 2 times for each sample.

After the equilibration period, 1.5 mL of the target malodor standard vapor is injected into each 10 mL vial. For thiol analysis, the vials containing a sample+malodor standard are held at room temperature for 30 minutes. Then, a 1 mL headspace syringe is then used to inject 250 μL of each sample/malodor into a GC/MS split/splitless inlet. A GC pillow is used for the amine analysis to shorten the run times.

Samples are then analyzed using a GC/MS with a DB-5, 20 m, 1 μm film thickness column with an MPS-2 autosampler equipment with static headspace function. Data is analyzed by ion extraction on each total ion current (56 for thiol) and the area is used to calculate the percent reduction from the malodor standard for each sample.

Results of the GC/MS analysis are shown in Table 7. Here, the results are reported as % reduction, and higher numbers represent higher reduction in butanethiol concentration. This demonstrates the efficacy of the present invention in reducing thiol malodor in the vapor phase.

TABLE 7

| | | Catalyst | | |
|---|---|---|---|---|
| | | 1% | 1% | 1% |
| | No | w/w n- | w/w n- | w/w |
| Activated Alkene | catalyst | octylamine | hexylamine | DBU |
| Diethyl Fumarate | 20.7 | 11.2 | 3.8 | 63 |
| n-hexyl acrylate | 31.9 | 21 | 11.2 | 23.1 |
| n-phenylmaleimide | 12.4 | 2.2 | 1.5 | 10 |
| Delta Damascone | 21 | 24.4 | 30.7 | 99.6 |
| N,N dimethylacrylamide- | 28.2 | 22.3 | 50 | 95.2 |
| 4- methoxy phenol | | | | |
| Diethylmaleate | 24.5 | 8.1 | 13.3 | 44.3 |

### Example 4

### Fabric and Air Refreshing Composition Against Garlic Malodor

A fabric refresher composition is prepared with and without activated alkene (diethyl maleate) and catalyst (n-octyl amine), according to the compositions shown in Table 8.

TABLE 8

| | Control | |
|---|---|---|
| Ingredient | composition | Composition 8 |
| Deionized Water | 95.887 | 95.888 |
| Ethanol | 3.0 | 3.247 |
| Lupasol HF+ | 0.0650 | |
| Diethylene Glycol | 0.175 | 0.250 |
| Silwet L-7600 | 0.1 | 0.100 |
| Maleic Acid | 0.05 | |
| Koralone B-119 | 0.015 | |
| Hydroxypropyl β-cyclodextrin | 0.630 | |
| Sodium Hydroxide | 0.003 | |
| Activated Alkene and Catalyst | | |
| (Premixed): | | |
| Diethyl Maleate | 0 | 0.483 |
| n-Octyl Amine | 0 | 0.032 |
| Total | | 100.000 |

+Available from BASF

To determine malodor reduction efficacy of the fabric refreshing malodor control composition in Table 8, malodor is first prepared according to the following procedure.

An electric skillet with temperature control is placed into a fume hood and set to 250° F. 80 grams of Crisco® oil are placed in the skillet which is then covered with a skillet lid. After 10 minutes for equilibration, the skillet lid is removed and the oil temperature is measured with a thermometer to ensure it is at 250° F. 50 grams of chopped, commercially prepared garlic in water are then placed into the skillet, and it is again covered with a lid. The garlic is cooked for 2.5 minutes or until garlic is translucent, with a portion staring to turn brown but not burn. Garlic is then removed from the skillet 5 grams of garlic are placed in each of 3 Petri dishes. Covers are placed on each Petri dish.

Malodor reduction efficacy is tested in a test chamber. Each test chamber is 39.25 inches wide, by 25 inches deep, by 21.5 inches high with a volume of 12.2 cubic feet (0.34 cubic meters). The test chamber can be purchased from Electro-Tech Systems, Glenside, Pa. Each test chamber is equipped with a fan (Newark catalog #70K9932, 115 VAC, 90CFM) purchased from Newark Electronics, Chicago, Ill.

Each previously prepared covered Petri dish, with 5 grams of garlic, is placed into an individual test chamber in front of the fan. The lids of the Petri dishes are then removed to expose contents for a dwell time sufficient to provide an initial odor intensity grade of 70-80 (about 2 minutes), as measured by trained panelists according to the scale shown in Table 9. Once the initial odor intensity grade has been reached in a test chamber, the Petri dish is removed from the test chamber.

Approximately 1.4 g of Composition 8 is then sprayed into the malodorous test chamber. For the malodor-only chamber, no composition is sprayed.

At pre-determined time intervals, trained evaluators open each chamber, smell the chamber for odor intensity, and assign a score for Malodor Intensity, based on the scale in Table 9. Immediately following, the trained evaluator smells the same chamber for perfume scent intensity, and assigns a score for scent intensity, also based on the scale in Table 9. The chamber door is closed between sequential evaluators. The scores are tabulated and the average malodor intensity and scent intensity scores for each time interval are recorded.

TABLE 9
Expert Sensory Grader Odor Evaluation Scale

| Score | Description corresponding to Score |
|---|---|
| 0 | No odor present |
| 10 | Very slight odor - “I think there is an odor |
| | present” |
| 20 | Slight odor - “I detect something but cannot |
| | identify specific odor |
| 25 | Slight odor |
| 50 | Moderate |
| 75 | Strong odor |
| 100 | Extremely Strong odor |

The malodor intensity according the scale in Table 9 is recorded 5, 20, 35, and 50 minutes after removal of the garlic-containing petri dish. Table 10 shows that Composition 8 reduces the intensity of garlic malodor further than the Control composition.

TABLE 10

| Time | Garlic | Control | |
|---|---|---|---|
| (minutes) | Malodor Only | composition | Composition 8 |
| 5 | 83 | 38 | 35 |
| 20 | 75 | 47 | 30 |
| 35 | 73 | 43 | 20 |
| 50 | 67 | 37 | 16 |

### Example 5

### Non-Energized Air Freshening Composition Against Garlic Malodor

A malodor reducing composition is prepared for use in a non-energized air freshening device according to Table 11 (“Composition 11”).

TABLE 11

| Ingredient | Purpose | wt. % |
|---|---|---|
| Dioctyl adipate | Diluent | 90.0 |
| Diethyl Maleate | Activated Alkene Malodor Control | 9.3 |
| n-Octyl Amine | Catalyst | 0.7 |
| Total | | 100.0 |

To prepare the non-energized air freshener, 5 grams of Composition 11 is placed into an empty Febreze® Set & Refresh air freshener utilizing a microporous membrane (Teslin 1100HD, PPG Industries Monroeville, Pa.) with a surface area of approximately 34 cm². Samples are tested 1 to 24 hours after activation (i.e when a test composition is allowed to contact the membrane), to ensure that the microporous membrane is fully saturated.

Garlic malodor is prepared as in Example 4.

Malodor reduction efficacy of the non-energized air freshener is tested in a test chamber. Each test chamber is 39.25 inches wide, by 25 inches deep, by 21.5 inches high with a volume of 12.2 cubic feet (0.34 cubic meters). The test chamber can be purchased from Electro-Tech Systems, Glenside, Pa. Each test chamber is equipped with a fan (Newark catalog #70K9932, 115 VAC, 90CFM) purchased from Newark Electronics, Chicago, Ill.

The Febreze® Set & Refresh air freshener with Composition 11, as described above, is introduced into the test chamber 5 minutes before the cooked garlic malodor. Each passive air freshener is placed into an individual test chambers on the opposite side of a small fan.

Each covered Petri dish (containing 5 grams of garlic) is placed into an individual test chamber in front of the fan. Note: One test chamber will not contain a passive dispenser device. This chamber will serve as the control chamber. The lids of the Petri dishes are removed to expose contents for a dwell time sufficient to provide an initial odor intensity grade of 70-80 in the control chamber (about 2 minutes), as measured by trained panelists according to the scale shown in Table 9. Once the initial odor intensity grade has been reached in the control chamber, the Petri dishes are removed from all of the test chambers.

At pre-determined time intervals, trained evaluators open each chamber, smell the chamber for odor intensity, and assign a score for Malodor Intensity, based on the scale in Table 9. The chamber door is closed between sequential evaluators. The scores are tabulated and the average malodor intensity and scent intensity scores for each time interval are calculated and recorded.

As shown in Table 12, the non-energized air freshener containing the malodor reducing composition of the current invention, Composition 11, significantly reduces malodor intensity whilst being substantially free of perfume raw materials.

TABLE 12

| | Garlic | |
|---|---|---|
| Time | Malodor Only | Composition |
| (minutes) | Control | 11 |
| 5 | 79 | 54 |
| 15 | 77 | 36 |
| 20 | 73 | 29 |
| 30 | 67 | 21 |
| 45 | 60 | 15 |
| 60 | 52 | 12 |

### Example 6

### Combination of Activated Alkene And Polyamine Polymer

This example illustrates the preparation and performance of a composition in accordance with the present invention containing a combination of a polyamine polymer and an activated alkene.

To create Compositions 13A-13C, a 50 ml mixture of water, ethanol, Silwet L-7600 surfactant and hexyl acrylate is prepared by mixing. Separately, a 50 ml aqueous solution of zinc polymer coordination complexes were prepared by stifling 0.2% ZnCl2 and 0.5% polymer for 30 minutes in water. Finally the solutions were combined and the solution pH was adjusted to 7 using 30% maleic acid. A blank solution (pH 7) was used as a representative Control.

TABLE 13

| | | Composi- | Composi- | Composi- |
|---|---|---|---|---|
| | | tion 13A | tion 13B | tion 13C |
| Ingredients | Control | (wt. %) | (wt. %) | (wt. %) |
| Ethanol | 3.0 | 3.0 | 3.0 | 3.0 |
| Surfactant (Silwet | 0.1 | 0.1 | 0.1 | 0.1 |
| 7600) | | | | |
| Hexyl acrylate | | 0.5 | | 0.25 |
| Zinc Lupamin | | | 0.7 | 0.5 |
| complex | | | | |
| Maleic Acid | As needed | As needed | As needed | As needed |
| Sodium Hydroxide | As needed | As needed | As needed | As needed |
| Water | balance | balance | balance | balance |
| Final pH | 7 | 7 | 7 | 7 |

5 ml of each Composition in Table 13 is placed in a GC-MS vial and spiked with 5 microliters of chemical surrogates shown in Table 13. The solutions are first equilibrated at room temperature for 2 hours, then incubated at 35° C. for 30 minutes. The headspace of each vial is finally sampled using a PDMS/SPME fiber and analyzed by GC/MS. The reductions in head space concentrations of odor molecules are measured and the data are normalized to Control.

Results of the SPME GC/MS analysis are shown in Table 14. Here the results are presented as % reduction compared to the control. Lower numbers denote high levels of malodor molecules present in the solution. Table 14 demonstrates that Composition 13C, comprising a combination of polyamine polymer and activated alkene, reduces a broad range of malodors effectively, with no negative interactions seen between the hexyl acrylate activated alkene and the polyamine polymer, except in the case of skatole.

TABLE 14

| | Odors | | | | | | | | | |
|---|---|---|---|---|---|---|---|---|---|---|
| | Methyl- | Pyri- | Octan- | Nonan- | | Butyl | Leaf | Dipropyl | Isovaleric | Butane- |
| Samples | pyrrole | dine | al | al | skatole | amine | alcohol | sulfide | acid | thiol |
| Control | 0 | 0 | 0 | 0 | 0 | 0 | 0 | 0 | 0 | 0 |
| Composition | 55 | 40 | 85 | 85 | 60 | 45 | 45 | 80 | 35 | 90 |
| 13A | | | | | | | | | | |
| Composition | 0 | 20 | 100 | 100 | 20 | 100 | 0 | 0 | 90 | 90 |
| 13B | | | | | | | | | | |
| Composition | 45 | 40 | 100 | 100 | 30 | 100 | 45 | 75 | 90 | 90 |
| 13C | | | | | | | | | | |

Throughout this specification, components referred to in the singular are to be understood as referring to both a single or plural of such component.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is, therefore, intended to cover in the appended claims all such changes and modifications that are within the scope of this invention.

What is claimed is:

1. A malodor control composition comprising:
an activated alkene; and
an organic catalyst;
wherein said composition is substantially free of mercaptans.

2. The malodor control composition of claim 1 wherein said activated alkene is selected from the group consisting of: a maleimide, an acrylic acid ester, a methacylic acid ester, a fumaric acid ester, a maleic acid ester, an acrylonitrile, an α, β unsaturated ketone, an α, β unsaturated aldehyde, and mixtures thereof, preferably said activated alkene is an α, β unsaturated carbonyl.

3. The malodor control composition of claim 1 wherein said activated alkene is present in an amount from 0.005% to 50%, by weight of said composition, preferably said activated alkene is present in an amount from 0.05% to 25%, by weight of said composition.

4. The malodor control composition according to any one of the preceding claims wherein said catalyst is selected from the group consisting of: a primary amine, a secondary amine, a phosphine, a diazo compound, and mixtures thereof, preferably said catalyst is a diazo compound.

5. The malodor control composition according to any one of the preceding claims wherein the molar ratio of said catalyst to said activated alkene is from 1:5 to 1:100,000, preferably the molar ratio of said catalyst to said activated alkene is from 1:10 to 1:250.

6. The malodor control composition according to any one of the preceding claims wherein said composition further comprises a polyamine polymer, preferably a metal coordinated polyamine polymer.

7. The malodor control composition according to any one of the preceding claims wherein said composition further comprises a perfume raw material, preferably said perfume raw material comprises a volatile aldehyde selected from the group consisting of 2-ethoxy benzylaldehyde, 2-isopropyl-5-methyl-2-hexenal, 5-methyl furfural, 5-methyl-thiophene-carboxaldehyde, adoxal, p-anisaldehyde, benzylaldehyde, bourgenal, cinnamic aldehyde, cymal, decyl aldehyde, floral super, florhydral, helional, lauric aldehyde, ligustral, lyral, melonal, o-anisaldehyde, pino acetaldehyde, P.T. bucinal, thiophene carboxaldehyde, trans-4-decenal, trans trans 2,4-nonadienal, undecyl aldehyde, and mixtures thereof.

8. The malodor control composition according to any one of the preceding claims wherein said composition has a pH of 4 to 7.

9. The malodor control composition according to any one of the preceding claims wherein said composition has a VP from 0.001 torr to 0.5 torr measured at 25° C.

10. The malodor control composition of claim 1 wherein said composition comprises less than 10%, by weight of said composition, of mercaptans, preferably said composition is free of mercaptans.

11. A method of neutralizing a malodor in the air or on inanimate surfaces comprising:
contacting said malodor with a composition comprising:
an activated alkene; and
an organic catalyst.

12. The method of claim 11 wherein said malodor is a thiol malodor.

13. The method of claim 11 wherein said composition is substantially free of a mercaptan.
